(19) United States
(12) Patent Application Publication
(10) Pub. No.: US 2026/0015343 A1
(43) Pub. Date: Jan. 15, 2026

(54) CRYSTALLINE FORM OF AFICAMTEN

(71) Applicant: ZYDUS LIFESCIENCES LIMITED, Gandhinagar (IN)

(72) Inventors: Kumar Kamlesh Singh, Gandhinagar (IN); Jitesh Amratlal Desai, Gandhinagar (GJ); Chirag Jayantilal Vyas, Gandhinagar (IN); Jaydev Rameshchandra Vaghela, Gandhinagar (IN); Rameshwar Vishwas Mahale, Gandhinagar (IN)

(21) Appl. No.: 19/261,140

(22) Filed: Jul. 7, 2025

(30) Foreign Application Priority Data
Jul. 12, 2024 (IN) 202421053430

(51) Int. Cl. C07D 413/12

(52) U.S. Cl. CPC C07D 413/12

(57) ABSTRACT

The present invention relates to crystalline Form of aficamten. In particular, the present invention relates to crystalline Form Z1 of aficamten and process for preparation thereof. The present invention also relates to a pharmaceutical composition comprising the crystalline Form Z1 of aficamten. The invention also relates to the stable crystalline Form Z1 of aficamten.

### CROSS REFERENCE TO RELATED APPLICATION

This application claim priority under 35 USC 119(a) to Indian Application No. 202421053430, filed Jul. 12, 2024, which is hereby incorporated by reference in its entirety.

### FIELD OF THE INVENTION

The present invention relates to crystalline Form of aficamten. In particular, the present invention relates to crystalline Form Z1 of aficamten and process for preparation thereof. The present invention also relates to a pharmaceutical composition comprising the crystalline Form Z1 of aficamten. The invention also relates to the stable crystalline Form Z1 of aficamten.

### BACKGROUND

The following discussion of the prior art is intended to present the invention in an appropriate technical context and allow its significance to be properly appreciated. Unless clearly indicated to the contrary, reference to any prior art in this specification should be construed as such art is widely known or forms part of common general knowledge in the field.

Aficamten is an investigational, oral, small molecule cardiac myosin inhibitor and is chemically known as N-[(1R)-5-(5-ethyl-1,2,4-oxadiazol-3-yl)-2,3-dihydro-1H-inden-1-yl]-1-methylpyrazole-4-carboxamide, having chemical structure as shown below:

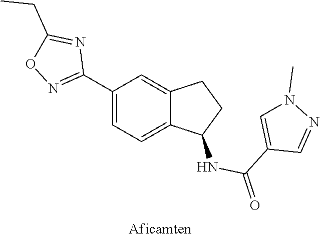 [SMILES: CCc1nc(-c2ccc3c(c2)CC[C@H]3NC(=O)c2cnn(C)c2)no1]

International (PCT) Publication No. 2021/011807 discloses polymorphic Form I, Form II, Form III, Form IV, Form V and Form VI of aficamten and process for their preparation.

Physical properties of the compound exhibited by polymorphs affect important pharmaceutical parameters for example stability, storage, density, compressibility and dissolution rates which are important for the determination of bioavailability. Stability differences may result in changes in chemical reactivity (for example hydrolysis or oxidation, such that a dosage form discolors more rapidly when comprised of one polymorph than when comprised of another polymorph), mechanical changes (for example tablets crumble on storage as a kinetically favored crystalline form converts to thermodynamically more stable crystalline Form).

The prior art has limited disclosure on polymorphic Forms of aficamten and their stability. Therefore, it is necessary to perform comprehensive polymorph screening of aficamten to select a crystalline Form which is stable and the most suitable for drug development.

### SUMMARY

In one general aspect, there is provided a crystalline Form of aficamten, designated as crystalline Form Z1.

In another general aspect, there is provided crystalline Form Z1 of aficamten characterized by having peaks expressed in degree(s) 2θ at about 3.8°, 11.2°, 12.9°, and 22.5°±0.2° with an absence of a peak at 14.4°±0.2°, when measured by X-ray powder diffraction and having a chemical purity of about 99.5% or more, when measured by area percentage of HPLC.

In another general aspect, there is provided the crystalline Form Z1 of aficamten wherein the crystalline Form Z1 is further characterized by peaks expressed in degree(s) 2θ at about 3.8°, 11.2°, 12.9°, 13.6°, 18.7°, 22.5°, 25.9°, 26.3°±0.2°, when measured by X-ray powder diffraction.

In another general aspect, there is provided a crystalline Form Z1 of aficamten, wherein the crystalline Form Z1 has a chemical purity of about 99.7% or more, when measured by area percentage of HPLC.

In another general aspect, there is provided a crystalline Form Z1, wherein the crystalline Form Z1 of aficamten is further characterized by a differential scanning calorimetry (DSC) having an endothermic peak at about 200±3° C. at a heating rate of 10° C./min.

In another general aspect, there is provided a crystalline Form Z1 of aficamten, wherein the crystalline Form Z1 remains stable for at least about 6 months when stored at 40° C./75% RH and which does not convert into any other crystalline form or an amorphous form.

In another general aspect, there is provided a crystalline Form Z1 of aficamten, wherein the crystalline Form Z1 has a chemical purity of about 99.5% or more, when measured by area percentage of HPLC for at least about 6 months, when stored at 40° C./75% RH.

In another general aspect, there is provided, a crystalline form Z1 of aficamten, wherein the crystalline Form Z1 has a chemical purity of about 99.7% or more, when measured by area percentage of HPLC, for at least about 6 months, when stored at 40° C./75% RH.

In another general aspect, there is provided a crystalline Form Z1 of aficamten having total impurities of about 0.5% or less, and single individual impurity of about 0.15% or less, when measured by area percentage of HPLC.

In another general aspect, there is provided a process for the preparation of a crystalline Form Z1 of aficamten, the process comprising:
(a) treating aficamten with a solvent or a mixture of solvents; and
(b) obtaining the crystalline Form Z1 of aficamten by the removal of the solvent.

In another general aspect, there is provided a process for the preparation of a crystalline Form Z1 of aficamten, the process comprising:
a) treating aficamten with a solvent or a mixture of solvents to obtain a reaction mixture;
b) heating the reaction mixture at 60-70° C.;
c) cooling the reaction mixture to 50-55° C.;
d) maintaining the reaction mixture for sufficient time and cooling to 0-15° C.; and
e) obtaining the crystalline Form Z1 of aficamten by the removal of the solvent.

In another general aspect, there is provided a composition comprising crystalline Form Z1 of aficamten characterized by having peaks expressed in degree(s) 2θ at about 3.8°, 11.2°, 12.9°, and 22.5°±0.2° with an absence of a peak at 14.4°±0.2°, when measured by X-ray powder diffraction.

In another general aspect, there is provided a composition comprising crystalline Form Z1 of aficamten, wherein the composition is a pharmaceutical composition together with one or more pharmaceutically acceptable carriers, excipients or diluents.

In another general aspect, there is provided a composition comprising crystalline Form Z1 of aficamten, wherein the crystalline Form Z1 of aficamten is having total impurities of about 0.5% or less for at least 6 months, when stored at 40° C./75% RH, when measured by area percentage of HPLC.

In another general aspect, there is provided a composition comprising crystalline Form Z1 of aficamten, wherein the crystalline Form Z1 of aficamten has a chemical purity of about 99.5% or more, and single individual impurity of about 0.15% or less, when measured by area percentage of HPLC.

In another general aspect, there is provided a crystalline Form Z1 of aficamten having particle size distributions wherein D10 is 50 μm or less, D50 is 200 μm or less, and D90 is 400 μm or less, or any combination thereof, as measured by Malvern Light Scattering method.

In another general aspect, there is provided a crystalline Form Z1 of aficamten having particle size distributions wherein D10 is about 50 μm or less, about 25 μm or less, and about 10 μm or less, D50 is about 200 μm or less, about 100 μm or less, and about 50 μm or less and D90 is about 400 μm or less, about 200 μm or less and about 100 μm or less, or any combination thereof, as measured by Malvern Light Scattering method.

In another general aspect, there is provided a stable crystalline Form Z1 of aficamten characterized by having peaks expressed in degree(s) 2θ at about 3.8°, 11.2°, 12.9°, and 22.5°±0.2° with an absence of a peak at 14.4°±0.2°, when measured by X-ray powder diffraction and having a chemical purity of about 99.5% or more, when measured by area percentage of HPLC.

In another general aspect, there is provided a pharmaceutical composition comprising crystalline Form Z1 of aficamten, wherein the crystalline Form Z1 is characterized by having peaks expressed in degree(s) 2θ at about 3.8°, 11.2°, 12.9°, and 22.5°±0.2°, when measured by X-ray powder diffraction and having a chemical purity of about 99.5% or more, when measured by area percentage of HPLC, together with one or more pharmaceutically acceptable carriers, excipients or diluents.

In another general aspect, there is provided a pharmaceutical composition comprising crystalline Form Z1 of aficamten, wherein the crystalline Form Z1 of aficamten remains stable for at least about 6 months, when stored at 40° C./75% RH and which does not convert into any other crystalline form or an amorphous form.

### BRIEF DESCRIPTION OF DRAWINGS

FIG. 1: X-ray powder diffraction (XRD) pattern of a crystalline Form Z1 of aficamten

FIG. 2: Differential scanning calorimetry (DSC) curve of a crystalline Form Z1 of aficamten

FIG. 3: Thermal gravimetric analysis (TGA) curve of a crystalline Form Z1 of Aficamten

### DETAILED DESCRIPTION OF THE INVENTION

The aforementioned general and further aspects of the invention are achieved by the detailed description of the invention provided herein after.

While the invention has been described in terms of its specific embodiments, the description, in no way, intends to limit the scope of the present invention to the specifically described embodiments only; equivalents and variants thereof which are apparently obvious to those skilled in the art are also included within the scope of the present invention. The description does not include detailed descriptions of conventional methods used in the field of the invention; such conventional methods are well known to those of ordinary skill in the art either because they are normally practiced routinely by the skilled artisan and/or are described in detail in various publications physical as well as digital.

The term “solution” as used herein, unless described otherwise, does not necessarily mean only a clear solution or one wherein the solute is completely soluble in the solvent; all the intermediate phases of mixture of components, starting from a state wherein the solute has stated getting dissolved in the solvent to a state wherein the solute has completely dissolved in the solvent, are included within the expression ‘solution’.

As used herein, the term “area percentage of HPLC” refers to the area percentage of a peak in HPLC (high performance liquid chromatography) chromatogram. In some embodiments, an area percentage is relative to the total area of the composition in a HPLC chromatogram. In some embodiments, an area percentage is relative to the area of aficamten in a HPLC chromatogram.

The terms ‘obtaining’, ‘isolating’, and ‘purifying’ are generally interchangeable and include, but not limited to, decantation, filtration, extraction, evaporation, crystallization, recrystallization and chromatographic operations.

As used herein, the term “stable crystalline Form Z1 of aficamten” includes crystalline aficamten that after exposure to a relative humidity of 75% at 40° C. or 60% at 25° C., for a period of at least six (6) months and doesn't change to any other crystalline form or an amorphous form.

As used herein, the term “sufficient time” means the amount of time, on average, reasonably required to complete the task with reasonable effectiveness or the duration needed for reactants to transform into products.

Optionally, the solution, prior to any solids formation and/or solvent removal, can be filtered to remove any undissolved solids, particulate matter or solid impurities. Any filtration system and filtration techniques known in the art can be used for the purpose.

All ranges recited herein include the endpoints, including those that recite a range “between” two values. Terms such as “about”, and “generally”, are to be construed as modifying a term or value to which they are attached such that the term or the value is not absolute. This includes, at very least, the degree of expected experimental error, technique error and instrument error for a given technique used to measure a value.

The term “pharmaceutically acceptable” as used herein is intended to mean that it is useful in preparing a pharmaceutical composition that is generally non-toxic and is not biologically undesirable and includes that which is acceptable for veterinary use and/or human pharmaceutical use.

The term “composition” used herein means a physical mixture of two or more components.

The term “pharmaceutical composition” is intended to encompass a drug product including the active ingredient(s), pharmaceutically acceptable excipients that make up the carrier, as well as any product, which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The product(s) obtained may further be purified to obtain them in purer form.

The product(s) obtained may further be dried additionally to achieve desired level of moisture and/or residual solvents.

The product(s) obtained may further be converted to any other physical forms thereof which includes but not specifically limited to polymorph(s), salt(s), solvate(s), hydrate(s), co-crystal(s) or solid dispersion(s); and crystalline or amorphous forms thereof.

The product(s) obtained may further be subjected to physical processing which includes, but not limited to, sieving, pressing, crushing, triturating, milling or grinding to adjust the particle size of the product(s) to desired levels.

The product(s) obtained may further be combined with pharmaceutically acceptable career to obtain a pharmaceutical composition comprising one or more of the solid-state forms of Aficamten of the invention and pharmaceutically acceptable carriers, excipients or diluents.

The pharmaceutical composition may be in the form of solid or liquid dosage forms and may have immediate release or modified release characteristics. The dosage forms include but are not limited to tablet, capsule, powder, granules, solution, suspension, emulsion, elixir or cream.

The compound aficamten used herein as starting material may be prepared by the known processes in the art, some of them are as disclosed in U.S. PG-Pub. No. 2025/0034095 A1, which is herein incorporated as reference in its entirety.

In one general aspect, there is provided a crystalline Form of aficamten, designated as crystalline Form Z1.

In another general aspect, there is provided crystalline Form Z1 of aficamten characterized by having peaks expressed in degree(s) 2θ at about 3.8°, 11.2°, 12.9°, and 22.5°±0.2° with an absence of a peak at 14.4°±0.2°, when measured by X-ray powder diffraction and having a chemical purity of about 99.5% or more, when measured by area percentage of HPLC.

In another general aspect, there is provided crystalline Form Z1 of aficamten wherein the crystalline Form Z1 is further characterized by peaks expressed in degree(s) 2θ at about 3.8°, 11.2°, 12.9°, 13.6°, 18.7°, 22.5°, 25.9°, 26.3°±0.2°, when measured by X-ray powder diffraction.

Furthermore, the X-ray powder diffraction pattern of the crystalline Form Z1 of aficamten shows peaks expressed in degree(s) 2θ at about 3.8°±0.2°, 11.2°±0.2°, 12.9°±0.2°, 13.6°±0.2°, 18.7°±0.2°, 22.5°±0.2°, 25.9°±0.2° and 26.3°±0.2° with absence of a peak at about 14.4°±0.20.

Table-1 shows peaks at 2θ°±0.2° for the crystalline Form Z1 of Aficamten.

TABLE 1
Degree 2-theta values (±0.2°)

| | |
|---|---|
| 3.77 | 22.48 |
| 7.50 | 23.17 |
| 10.11 | 24.04 |
| 11.23 | 24.39 |
| 12.90 | 24.84 |
| 13.57 | 25.93 |
| 14.96 | 26.28 |
| 15.46 | 27.23 |
| 16.72 | 28.31 |
| 17.90 | 29.17 |
| 17.99 | 30.15 |
| 18.71 | 31.43 |
| 19.45 | 33.96 |
| 20.73 | 36.23 |
| 20.93 | 36.77 |
| 21.52 | 37.65 |
| 22.19 | 37.99 |

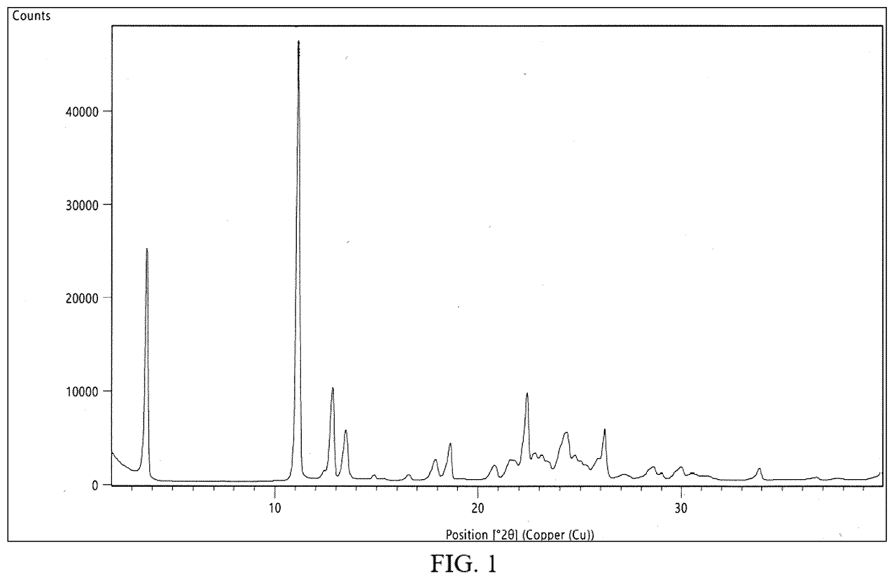

In another general aspect, the X-ray powder diffraction pattern of the crystalline Form Z1 of aficamten is substantially as same as depicted in FIG. 1.

In another general aspect, there is provided a crystalline Form Z1 of aficamten, wherein the crystalline Form Z1 has a chemical purity of about 99.7% or more, when measured by area percentage of HPLC.

In another general aspect, there is provided a crystalline Form Z1, wherein the crystalline Form Z1 of aficamten is further characterized by a differential scanning calorimetry (DSC) having an endothermic peak at about 200±3° C. at a heating rate of 10° C./min.

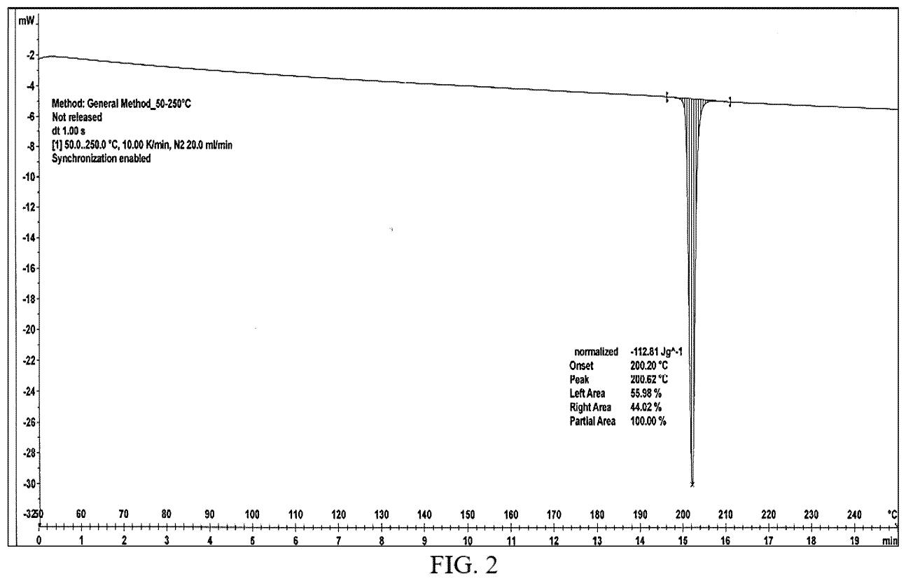

In another general aspect, the differential scanning calorimetry (DSC) curve of Form Z1 of aficamten is substantially as same as depicted in FIG. 2.

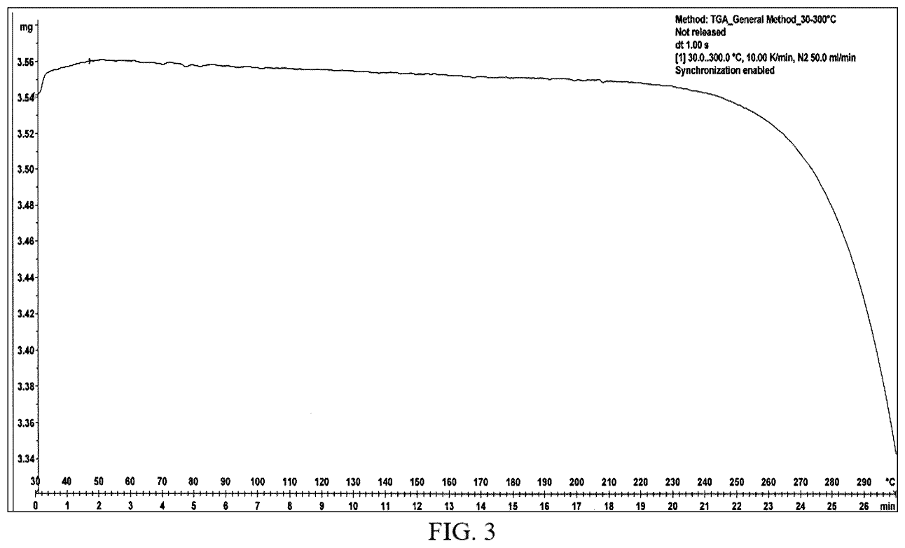

In another general aspect, the thermal gravimetric analysis (TGA) curve of crystalline Form Z1 of aficamten is substantially as same as depicted in FIG. 3.

In another general aspect, there is provided a crystalline Form Z1 of aficamten, wherein the crystalline Form Z1 remains stable for at least about 6 months when stored at 40° C./75% RH and which does not convert into any other crystalline form or an amorphous form.

In general, any other crystalline form of aficamten includes crystalline Form I, Form II, Form III, Form IV, Form V, and Form VI disclosed in International (PCT) Publication No. 2021/011807.

In another general aspect, there is provided a crystalline Form Z1 of aficamten, wherein the crystalline Form Z1 has a chemical purity of about 99.5% or more, when measured by area percentage of HPLC for at least about 6 months, when stored at 40° C./75% RH.

In general, the crystalline Form Z1 of aficamten of the invention is stable during drying and storage.

In general, the following Table-2 provides the stability data of crystalline Form Z1 of aficamten for upto 3 months.

TABLE 2

| | | | 1 Month | 2 Month | 3 Month | |
|---|---|---|---|---|---|---|
| Sr. | | | 25° C./ | 25° C./ | 25° C./ | 40° C./ |
| No. | Specification | Initial | 60% RH | 60% RH | 60% RH | 75% RH |
| 1 | Appearance | White to off- | White | White | White | White |
| | | white color solid | | | | |
| 2 | Polymorph | Crystalline | Crystalline | Crystalline | Crystalline | Crystalline |
| | | Form Z1 | Form Z1 | Form Z1 | Form Z1 | Form Z1 |
| 3 | Purity | 99.99% | >99% | >99% | >99% | >99% |

In general, the following Table-3 provides the stability data of crystalline Form Z1 of aficamten for up to 6 months at 40° C./75% RH.

TABLE 3

| Stability | | | 1 Month | 2 Month | 3 Month | 6 Month |
|---|---|---|---|---|---|---|
| station | | | 40° C./ | 40° C./ | 40° C./ | 40° C./ |
| Tests | Specification | Initial | 75% RH | 75% RH | 75% RH | 75% RH |
| Description | White to | White | White | White | White | White |
| | off-white | | | | | |
| | color solid | | | | | |

Related substances by HPLC (% w/w)

| | | | | | | |
|---|---|---|---|---|---|---|
| Any | NMT 0.10 | 0.02 | 0.03 | 0.03 | 0.03 | 0.03 |
| unspecified | | | | | | |
| impurity | | | | | | |
| Total | NMT 1.0 | 0.02 | 0.03 | 0.03 | 0.03 | 0.04 |
| impurities | | | | | | |
| Assay by | NLT 98 to | 100.1 | 100.1 | 100.1 | 99.9 | 99.9 |
| HPLC (% | NMT102.0 | | | | | |
| w/w, ODB) | | | | | | |
| (S)-Isomer | NMT 0.15 | ND | ND | ND | ND | ND |
| content by | | | | | | |
| HPLC | | | | | | |
| (% w/w) | | | | | | |
| Polymorph | Crystalline | Crystalline | Crystalline | Crystalline | Crystalline | Crystalline |
| by XRD | Form Z1 | Form Z1 | Form Z1 | Form Z1 | Form Z1 | Form Z1 |

Abbreviations—NLT: Not less than;
NMT: Not more than;
% w/w: Weight by weight;
ODB: On dried basis

In another general aspect, there is provided a crystalline form Z1 of aficamten, wherein the crystalline Form Z1 has a chemical purity of about 99.7% or more, when measured by area percentage of HPLC, for at least about 6 months when stored at 40° C./75% RH.

In another general aspect, there is provided a crystalline Form Z1 of aficamten having total impurities of about 0.5% or less, and single individual impurity of about 0.15% or less, when measured by area percentage of HPLC.

In general, the single individual impurity is of about 0.10% or less, when measured by area percentage of HPLC.

In another general aspect, there is provided a process for the preparation of crystalline Form Z1 of aficamten, the process comprising:
(a) treating aficamten with a solvent or a mixture of solvents; and
(b) obtaining the crystalline Form Z1 of aficamten by the removal of the solvent.

In general, the solvent or mixture of solvents at step (a) is selected from one or more water, methanol, ethanol, isopropanol, n-butanol, acetone, methyl ethyl ketone, methyl isobutyl ketone, acetonitrile, dimethyl formamide, N,N-dimethyl acetamide, dimethyl sulfoxide, and methylene dichloride, or a mixture thereof. Particularly, the solvent is methanol, water or a mixture thereof.

In another general aspect, there is provided a process for the preparation of a crystalline Form Z1 of aficamten, the process comprising:
a) treating aficamten with a solvent or a mixture of solvents to obtain a reaction mixture; b) heating the reaction mixture at 60-70° C.;
c) cooling the reaction mixture to 50-55° C.;
d) maintaining the reaction mixture for sufficient time and cooling to 0-15° C.; and e) obtaining the crystalline Form Z1 of aficamten by the removal of the solvent.

In general, the solvent or mixture of solvents at step (a) is selected from methanol, water or mixture thereof.

The reaction may be maintained for sufficient time i.e. for the time reasonably required to complete the reaction effectively or the duration needed for reactants to transform into products.

The solvent can be removed by techniques known in the art such as filtration, filtration under vacuum, decantation, simple distillation, and distillation under vacuum, centrifugation or any other suitable technique.

In another general aspect, there is provided a process for the preparing crystalline Form Z1 of aficamten, the process comprising:
(a) treating aficamten with methanol, water, or a mixture thereof; and
(b) obtaining the crystalline Form Z1 of aficamten by the removal of solvent.

In general, the reaction at step (a), wherein aficamten is treated with 5-10 volumes of methanol and 0-3 volumes of water. In particular, the water may be from 0.25-2.5 volumes, more particularly, 0.25-2.0 volumes may be used.

In general, the process for the preparation of crystalline Form Z1 of aficamten further comprises of addition of seed crystals during the crystallization, which is exemplified in one or more examples herein after.

In general, suitable techniques which may be used for the removal of solvent from the solution to obtain crystalline Form Z1 of aficamten at step (b), comprises one or more of filtration, filtration under vacuum, decantation, distillation, distillation at reduced pressure, centrifugation, etc. or any other suitable technique.

In general, the crystalline Form Z1 of aficamten of the invention has a purity of at least about 98% by area percentage of HPLC, preferably at least 99% by area percentage of HPLC, more preferably at least 99.5% by area percentage of HPLC, most preferably at least 99.7% by area percentage of HPLC.

In another general aspect, there is provided a composition comprising crystalline Form Z1 of aficamten characterized by having peaks expressed in degree(s) 2θ at about 3.8°, 11.2°, 12.9°, and 22.5°±0.2° with an absence of a peak at 14.4°±0.2°, when measured by X-ray powder diffraction.

In another general aspect, there is provided a composition comprising crystalline Form Z1 of aficamten, wherein the composition is a pharmaceutical composition together with one or more pharmaceutically acceptable carriers, excipients or diluents.

In another general aspect, there is provided a composition comprising crystalline Form Z1 of aficamten, wherein the crystalline Form Z1 of aficamten is having total impurities of about 0.5% or less for at least 6 months, when stored at 40° C./75% RH, when measured by area percentage of HPLC.

In another general aspect, there is provided a composition comprising crystalline Form Z1 of aficamten, wherein the crystalline Form Z1 of aficamten has a chemical purity of about 99.5% or more, and single individual impurity of about 0.15% or less, when measured by area percentage of HPLC.

In another general aspect, there is provided a process for the preparation of a composition comprising crystalline Form Z1 of aficamten and a pharmaceutically acceptable carrier the process comprising:
(a) contacting crystalline Form Z1 of aficamten and a pharmaceutically acceptable carrier in one or more solvents to obtain a solution; and
(b) obtaining the solid composition of crystalline Form Z1 of aficamten and a pharmaceutically acceptable carrier by the removal of the solvent.

In general, the reaction at step (a) may be performed in the presence of solvent selected from water, methanol, ethanol, isopropanol, n-butanol, acetone, methyl ethyl ketone, methyl isobutyl ketone, acetonitrile, dimethyl formamide, N,N-dimethyl acetamide, dimethyl sulfoxide, methylene dichloride, or a mixture thereof.

In general, suitable techniques which may be used for the removal of solvent to obtain a composition comprising crystalline Form Z1 of aficamten and a pharmaceutically acceptable carrier, comprises one or more of filtration, filtration under vacuum, decantation, distillation, distillation at reduced pressure, centrifugation, etc. or any other suitable technique.

In another general aspect, the solution of aficamten can be evaporated to dryness to obtain stable crystalline Form Z1 of aficamten.

In another general aspect, there is provided a crystalline Form Z1 of aficamten having a chemical purity of about 98% or more, or about 99% or more, or about 99.5% or more, or about 99.7% or more, or about 99.9% or more, when measured by area percentage of HPLC.

X-ray powder diffraction pattern in the present invention is acquired by a PANalytical Empyrean X-ray powder diffractometer. The parameters of the X-ray powder diffraction method of the present invention are as follows:
X-ray Reflection: Cu, Kα
Kα1 (Å): 1.54060; Kα2 (Å): 1.54443
Kα2/Kα1 intensity ratio: 0.50
Voltage: 45 (kV)
Current: 40 (mA)
Scan range: from 2.0 degree to 40.0 degree.

Differential scanning calorimetry (DSC) data in the present invention are acquired by a Mettler Toledo DSC 3. The parameters of the differential scanning calorimetry (DSC) method of the present disclosure were as follow:
Heating rate: 10° C./min
Purge gas: nitrogen.

Thermal gravimetric analysis (TGA) data in the present invention are acquired by a Mettler Toledo TGA 2. The parameters of the thermal gravimetric analysis (TGA) method of the present disclosure were as follow:
Heating rate: 10° C./min
Purge gas: nitrogen.

In another general aspect, there is provided a crystalline Form Z1 of aficamten having particle size distributions wherein D10 is 50 μm or less, D50 is 200 μm or less, and D90 is 400 μm or less, or any combination thereof, as measured by Malvern Light Scattering method.

In another general aspect, there is provided a crystalline Form Z1 of aficamten having particle size distributions wherein D10 is about 50 μm or less, about 25 μm or less, and about 10 μm or less, D50 is about 200 μm or less, about 100 μm or less, and about 50 μm or less and D90 is about 400 μm or less, about 200 μm or less and about 100 μm or less, or any combination thereof, as measured by Malvern Light Scattering method.

In another general aspect, there is provided a crystalline Form Z1 of aficamten, further having particle size distributions wherein D10 is about 10 μm or less, D50 is about 25 μm or less and D90 is about 100 μm or less, or any combination thereof, as measured by Malvern Light Scattering method.

In general, the crystalline Form Z1 of aficamten may be micronized or sieved through specific mess size to further achieve the better particle size distribution in order to make suitable Formulation. In particular, the mess size of 10 μm or 20 μm or 40 μm, etc. may be used for sieving.

The particle size distribution test in the present invention is acquired by the Malvern 3000 The test is carried out by wet process, and the dispersion medium is 0.1% tween 80 in water. The parameters are as follow:

| | |
|---|---|
| Size distribution: Volume | Run Time: 10 s |
| Dispersion medium: 0.1% | Particle coordinates: Standard |
| tween 80 in water | |
| Run Number: Average of 3 runs | Fluid refractive index: 1.42 |
| Particle Transparency: Trans | Residuals: Enabled |
| Particle refractive index: 1.5 | Filtration: Enabled |
| Particle shape: Irregular | Ultrasonication time: 30 s |
| Ultrasonication power: 30 W | |

In another general aspect, there is provided a stable crystalline Form Z1 of aficamten characterized by having peaks expressed in degree(s) 2θ at about 3.8°, 11.2°, 12.9°, and 22.5°±0.2° with an absence of a peak at 14.4°±0.2°, when measured by X-ray powder diffraction and having a chemical purity of about 99.5% or more, when measured by area percentage of HPLC.

In another general aspect, there is provided a pharmaceutical composition comprising crystalline Form Z1 of aficamten, wherein the crystalline Form Z1 is characterized by having peaks expressed in degree(s) 2θ at about 3.8°, 11.2°, 12.9°, and 22.5°±0.2°, when measured by X-ray powder diffraction and having a chemical purity of about 99.5% or more, when measured by area percentage of HPLC, together with one or more pharmaceutically acceptable carriers, excipients or diluents.

In another general aspect, there is provided a pharmaceutical composition comprising crystalline Form Z1 of aficamten, wherein the crystalline Form Z1 of aficamten remains stable for at least about 6 months, when stored at 40° C./75% RH and which does not convert into any other crystalline form or an amorphous form.

In another general aspect, there is provided a pharmaceutical composition comprising crystalline Form Z1 of aficamten, wherein aficamten contains less than 0.5% (w/w) of total impurities during stability for at least about 6 months.

In another general aspect, there is provided a solid pharmaceutical formulation comprising aficamten in the crystalline state, wherein said solid pharmaceutical formulation is orally administrable and wherein the crystalline Form Z1 is stable and does not convert into other crystalline form or an amorphous form.

The present invention is further illustrated by the following reaction examples which are provided merely to be representative of the invention and do not limit the scope of it.

### EXAMPLES: PREPARATION OF CRYSTALLINE FORM Z1 OF AFICAMTEN

### Example 1

In a Round bottom flask, aficamten (1 g, 0.003 mole) was dissolved in methanol (7 ml) at reflux temperature under stirring. Stirring was stopped and the solution was cooled to 25-35° C. till solid precipitated out. Further it was cooled to 0-10° C. and maintained for 1 hour. The product was filtered and dried under vacuum to obtain crystalline Form Z1 of aficamten (0.8-0.9 g). HPLC purity: 99.96% EXAMPLE 2

In a Round bottom flask, aficamten (1 g, 0.003 mole) was dissolved in mixture of methanol (7 ml) and water (0.25 ml) at reflux temperature under stirring. Stirring was stopped and the solution was cooled to 25-35° C. or till solid precipitated out. The stirring was started, and the solution was further cooled to 0-10° C. and maintained for 1 hour. The product was filtered and dried under vacuum to obtain crystalline Form Z1 of aficamten (0.8-0.9 g). HPLC purity: 99.95%.

### Example 3

In a Round bottom flask, aficamten (1 g, 0.003 mole) was dissolved in mixture of methanol (7 ml) and water (0.5 ml) at reflux temperature under stirring. The solution was cooled to 25-35° C. or till solid precipitated out. The stirring was started, the solution was further cooled to 0-10° C. and maintained for 1 hour. The product was filtered and dried under vacuum to obtain crystalline Form Z1 of aficamten (0.8-0.9 g). HPLC purity: 100%

### Example 4

In a Round bottom flask, aficamten (5 g, 0.01 mole) was dissolved in mixture of methanol (7 ml) and water (5 ml) at reflux temperature under stirring. The solution to 25-35° C. or till solid precipitated out. The solution was cooled to 0-10° C. and maintained for 1 hour. The product was filtered and dried under vacuum to obtain crystalline Form Z1 of aficamten (4 to 4.5 g). HPLC purity: 99.98%

### Example 5

In a Round bottom flask, aficamten (1 g, 0.003 mole) was dissolved in mixture of methanol (7 ml) and water (2 ml) at reflux temperature under stirring. The solution was cooled to 25-35° C. or till solid precipitated out. The solution was further cooled to 0-10° C. and maintained for 1 hour. The product was filtered and dried under vacuum to obtain crystalline Form Z1 of aficamten (0.8-0.9 g). HPLC Purity: 99.96%

### Example 6

In a Round bottom flask, aficamten (1 g, 0.003 mole) was dissolved in methanol (7 ml) at reflux temperature under stirring. Water (1 ml) was added, and the solution was cooled to 25-35° C. or till solid precipitated out. The solution was further cooled to 0-10° C. and maintained for 1 hour. The product was filtered and dried under vacuum to obtain crystalline Form Z1 of aficamten (0.78 g). HPLC Purity: 99.99%.

### Example 7

In a Round bottom flask, 975 ml methanol was taken and 11 gm Aficamten was added with 25 ml methanol at 25-35° C. The reaction mixture was stirred at 60-70° C. to obtain a clear solution. Stirring was stopped and reaction mixture was cooled to 50-55° C. and seeded with 0.5 gm aficamten. After crystallization, the reaction mixture was cooled to 5-15° C. and stirred for 30 minutes at 5-15° C. The product was filtered under vacuum and the wet cake was washed using 100 ml methanol at 5-15° C. The product was then dried in VTD (Vacuum NLT 650 mmHg) for 5 to 6 hours to obtain aficamten Form Z1. Yield: 85%, Purity by HPLC: 99.98%, Assay by HPLC: 100.1%.

While the present invention has been described in terms of a few specific embodiments, certain modifications, and equivalents thereof, will be apparent to those skilled in the art and are intended to be included within the scope of the invention.

1. Crystalline Form Z1 of aficamten characterized by having peaks expressed in degree(s) 2θ at about 3.8°, 11.2°, 12.9°, and 22.5°±0.2° with an absence of a peak at 14.4°±0.2°, when measured by X-ray powder diffraction and having a chemical purity of about 99.5% or more, when measured by area percentage of HPLC.

2. The crystalline Form Z1 of aficamten according to claim 1, wherein the crystalline Form Z1 is further characterized by peaks expressed in degree(s) 2θ at about 3.8°, 11.2°, 12.9°, 13.6°, 18.7°, 22.5°, 25.9°, 26.3°±0.2°, when measured by X-ray powder diffraction.

3. The crystalline Form Z1 of aficamten according to claim 1, wherein the crystalline Form Z1 has a chemical purity of about 99.7% or more, when measured by area percentage of HPLC.

4. The crystalline Form Z1 of aficamten according to claim 1, wherein the crystalline Form Z1 is further characterized by a differential scanning calorimetry (DSC) having an endothermic peak at about 200±3° C. at a heating rate of 10° C./min.

5. The crystalline Form Z1 of aficamten according to claim 1, wherein the crystalline Form Z1 remains stable for at least about 6 months, when stored at 40° C./75% RH and which does not convert into any other crystalline form or an amorphous form.

6. The crystalline Form Z1 of aficamten according to claim 1, wherein the crystalline Form Z1 has a chemical purity of about 99.5% or more, when measured by area percentage of HPLC, for at least about 6 months, when stored at 40° C./75% RH.

7. The crystalline Form Z1 of aficamten according to claim 6, wherein the crystalline Form Z1 has a chemical purity of about 99.7% or more, when measured by area percentage of HPLC, for at least about 6 months, when stored at 40° C./75% RH.

8. The crystalline Form Z1 of aficamten according to claim 1 having total impurities of about 0.5% or less, and a single individual impurity of about 0.15% or less, when measured by area percentage of HPLC.

9. A process for the preparation of a crystalline Form Z1 of aficamten, the process comprising:
(a) treating aficamten with a solvent or a mixture of solvents; and
(b) obtaining the crystalline Form Z1 of aficamten by the removal of the solvent.

10. The process according to claim 9, wherein the solvent is selected from one or more of water, methanol, ethanol, isopropanol, n-butanol, acetone, methyl ethyl ketone, methyl isobutyl ketone, acetonitrile, dimethyl formamide, dimethyl sulfoxide and methylene dichloride, or a mixture thereof.

11. The process according to claim 9, further comprising:
a) treating aficamten with a solvent or mixture of solvents to obtain a reaction mixture;
b) heating the reaction mixture to 60-70° C.;
c) cooling the reaction mixture to 50-55° C.;
d) maintaining the reaction mixture for sufficient time and cooling to 0-15° C.; and
e) obtaining the crystalline Form Z1 of aficamten by the removal of the solvent.

12. The process according to claim 11, wherein the solvent is selected from methanol, water, or a mixture thereof.

13. A composition comprising crystalline Form Z1 of aficamten characterized by having peaks expressed in degree(s) 2θ at about 3.8°, 11.2°, 12.9°, and 22.5° 0 0.2° with an absence of a peak at 14.4°±0.2°, when measured by X-ray powder diffraction.

14. The composition according to claim 13, wherein the composition is a pharmaceutical composition together with one or more pharmaceutically acceptable carriers, excipients, or diluents.

15. The composition according to claim 13, wherein the crystalline Form Z1 of aficamten is having total impurities of about 0.5% or less for at least 6 months, when stored at 40° C./75% RH, when measured by area percentage of HPLC.

16. The composition according to claim 15, wherein the crystalline Form Z1 of aficamten has a chemical purity of about 99.5% or more, and a single individual impurity of about 0.15% or less, when measured by area percentage of HPLC.

17. A crystalline Form Z1 of aficamten having particle size distributions wherein D10 is 50 μm or less, D50 is 200 μm or less, and D90 is 400 μm or less, or any combination thereof, as measured by Malvern Light Scattering method.

18. A stable crystalline Form Z1 of aficamten characterized by having peaks expressed in degree(s) 2θ at about 3.8°, 11.2°, 12.9°, and 22.5°±0.2° with an absence of a peak at 14.4°±0.2°, when measured by X-ray powder diffraction and having a chemical purity of about 99.5% or more, when measured by area percentage of HPLC.

19. A pharmaceutical composition comprising crystalline Form Z1 of aficamten, wherein the crystalline Form Z1 is characterized by having peaks expressed in degree(s) 2θ at about 3.8°, 11.2°, 12.9°, and 22.5°±0.2°, when measured by X-ray powder diffraction and having a chemical purity of about 99.5% or more, when measured by area percentage of HPLC, together with one or more pharmaceutically acceptable carriers, excipients or diluents.

20. The pharmaceutical composition according to claim 19, wherein the crystalline Form Z1 of aficamten remains stable for at least about 6 months, when stored at 40° C./75% RH and which does not convert into any other crystalline form or an amorphous form.
